(19) United States
(12) Patent Application Publication
(10) Pub. No.: US 2017/0151425 A1
(43) Pub. Date: Jun. 1, 2017

(54) METHOD AND APPARATUS FOR MEASURING THE DOSE REMAINING UPON A TRANSDERMAL DRUG DELIVERY DEVICE

(71) Applicant: BKR IP Holdco LLC, Broomall, PA (US)

(72) Inventor: Bruce K. Redding, JR., Broomall, PA (US)

(21) Appl. No.: 15/323,641

(22) PCT Filed: Jul. 6, 2015
(86) PCT No.: PCT/US15/39274

§ 371 Date: Jan. 3, 2017

Related U.S. Application Data
(60) Provisional application No. 62/125,837, filed on Feb. 2, 2015.
(60) Provisional application No. 61/999,589, filed on Aug. 1, 2014.
(60) Provisional application No. 61/998,790, filed on Jul. 9, 2014.
(60) Provisional application No. 61/998,788, filed on Jul. 9, 2014.
(60) Provisional application No. 61/998,683, filed on Jul. 7, 2014.
(60) Provisional application No. 61/998,623, filed on Jul. 3, 2014.
(60) Provisional application No. 61/998,622, filed on Jul. 3, 2014.
(60) Provisional application No. 61/998,624, filed on Jul. 3, 2014.

(30) Foreign Application Priority Data
Jul. 6, 2015 (US) PCT/US2015/039236
Jul. 6, 2015 (US) PCT/US2015/039264
Jul. 6, 2015 (US) PCT/US2015/039268

(51) Int. Cl. A61M 37/00

(52) U.S. Cl. CPC A61M 37/0092; A61M 2205/3375; A61M 2205/3386; A61M 2205/52; A61M 2205/8206; A61M 2205/502; A61M 2205/18; A61M 2205/581; A61M 2205/587; A61M 2205/50

(57) ABSTRACT

The invention is a sensor device attached to a transdermal patch or other form of transdermal delivery device (TDD) which is (1) capable of determining the quantity of drug remaining within the TDD and (2) determining the quantity or dose delivered from the TDD to a patient in real time. The preferred embodiment is a sensor capable of transmitting acoustical energy between an ultrasonic generator, through a medicine-containing TDD, providing a density measurement of the solution within the TDD and thereby calculating the dose remaining within the patch, and leading to a calculation of the dose delivered to the patient.

### PRIORITY CLAIM, CROSS REFERENCE TO RELATED APPLICATIONS AND INCORPORATION BY REFERENCE

This application is related to, claims priority under, and claims the benefit of the following provisional applications filed in the United States Patent and Trademark Office: “MODIFIED TRANSDERMAL DELIVERY PATCH WITH MULTIPLE ABSORBENT PADS”, Bruce K. Redding, Jr., filed on Jul. 3, 2014, and having Ser. No. 61/998,623: “MODIFIED TRANSDERMAL DELIVERY DEVICE OR PATCH AND METHOD OF DELIVERING INSULIN FROM SAID MODIFIED TRANSDERMAL DELIVERY DEVICE”, Bruce K. Redding, Jr., filed on Jul. 3, 2014, and having Ser. No. 61/998,622; “METHOD FOR GLUCOSE CONTROL IN DIABETICS”, Bruce K. Redding, Jr., filed on Jul. 3, 2014, and having Ser. No. 61/998,624; “ULTRASONIC TRANSDUCERS SUITABLE FOR ULTRASONIC DRUG DELIVERY VIA A SYSTEM WHICH IS PORTABLE AND WEARABLE BY THE PATIENT”, Bruce K. Redding, Jr., filed on Jul. 7, 2014, and having Ser. No. 61/998,683; “METHOD FOR THE ATTENUATION ENHANCEMENT OF ABSORBENT MATERIALS USED IN BOTH PASSIVE AND ACTIVE TRANSDERMAL DRUG DELIVERY SYSTEMS”, Bruce K. Redding, Jr., filed on Jul. 9, 2014, and having Ser. No. 61/998,788; “MODIFICATION OF PHARMACEUTICAL PREPARATIONS TO MAKE THEM MORE CONDUCIVE TO ULTRASONIC TRANSDERMAL DELIVERY”, Bruce K. Redding, Jr., filed on Jul. 9, 2014, and having Ser. No. 61/998,790; “METHOD AND APPARATUS FOR MEASURING THE DOSE REMAINING UPON A TRANSDERMAL DRUG DELIVERY DEVICE”, Bruce K. Redding, Jr., filed on Aug. 1, 2014, and having Ser. No. 61/999,589; “METHOD AND APPARATUS FOR EFFECTING ALTERNATING ULTRASONIC TRANSMISSIONS WITHOUT CAVITATION”, Bruce K. Redding, Jr., filed on Feb. 2, 2015, and having Ser. No. 62/125,837; PCT applications filed in the United States Patent and Trademark Office: “MODIFIED TRANSDERMAL DELIVERY PATCH WITH MULTIPLE ABSORBENT PADS”, Bruce K. Redding, Jr., filed on Jul. 6, 2015, and having serial number PCT/US15/39236; MODIFIED TRANSDERMAL DELIVERY DEVICE OR PATCH AND METHOD OF DELIVERING INSULIN FROM SAID MODIFIED TRANSDERMAL DELIVERY DEVICE, Bruce K. Redding, Jr., filed on Jul. 6, 2015, and having serial number PCT/US15/39264; METHOD FOR GLUCOSE CONTROL IN DIABETICS Bruce K. Redding, Jr., filed on Jul. 6, 2015, PCT/US15/39268.

This application hereby incorporates herein by reference the subject matter disclosed in the written descriptions, the drawings and claims, in their entireties of the following provisional applications filed in the United States Patent and Trademark Office: “MODIFIED TRANSDERMAL DELIVERY PATCH WITH MULTIPLE ABSORBENT PADS”, Bruce K. Redding, Jr., filed on Jul. 3, 2014, and having Ser. No. 61/998,623: “MODIFIED TRANSDERMAL DELIVERY DEVICE OR PATCH AND METHOD OF DELIVERING INSULIN FROM SAID MODIFIED TRANSDERMAL DELIVERY DEVICE”, Bruce K. Redding, Jr., filed on Jul. 3, 2014, and having Ser. No. 61/998,622; “METHOD FOR GLUCOSE CONTROL IN DIABETICS”, Bruce K. Redding, Jr., filed on Jul. 3, 2014, and having Ser. No. 61/998,624; “ULTRASONIC TRANSDUCERS SUITABLE FOR ULTRASONIC DRUG DELIVERY VIA A SYSTEM WHICH IS PORTABLE AND WEARABLE BY THE PATIENT”, Bruce K. Redding, Jr., filed on Jul. 7, 2014, and having Ser. No. 61/998,683; “METHOD FOR THE ATTENUATION ENHANCEMENT OF ABSORBENT MATERIALS USED IN BOTH PASSIVE AND ACTIVE TRANSDERMAL DRUG DELIVERY SYSTEMS”, Bruce K. Redding, Jr., filed on Jul. 9, 2014, and having Ser. No. 61/998,788; “MODIFICATION OF PHARMACEUTICAL PREPARATIONS TO MAKE THEM MORE CONDUCIVE TO ULTRASONIC TRANSDERMAL DELIVERY”, Bruce K. Redding, Jr., filed on Jul. 9, 2014, and having Ser. No. 61/998,790; “METHOD AND APPARATUS FOR MEASURING THE DOSE REMAINING UPON A TRANSDERMAL DRUG DELIVERY DEVICE”, Bruce K. Redding, Jr., filed on Aug. 1, 2014, and having Ser. No. 61/999,589; “METHOD AND APPARATUS FOR EFFECTING ALTERNATING ULTRASONIC TRANSMISSIONS WITHOUT CAVITATION”, Bruce K. Redding, Jr., filed on Feb. 2, 2015, and having Ser. No. 62/125,837; PCT applications filed in the United States Patent and Trademark Office: “MODIFIED TRANSDERMAL DELIVERY PATCH WITH MULTIPLE ABSORBENT PADS”, Bruce K. Redding, Jr., filed on Jul. 6, 2015, and having serial number PCT/US15/39236; MODIFIED TRANSDERMAL DELIVERY DEVICE OR PATCH AND METHOD OF DELIVERING INSULIN FROM SAID MODIFIED TRANSDERMAL DELIVERY DEVICE, Bruce K. Redding, Jr., filed on Jul. 6, 2015, and having serial number PCT/US15/39264; METHOD FOR GLUCOSE CONTROL IN DIABETICS Bruce K. Redding, Jr., filed on Jul. 6, 2015, PCT/US15/39268.

### FIELD OF THE INVENTION

The present invention relates generally to transdermal substance delivery, and more specifically to a sensor, which can be attached to a wearable transdermal drug delivery device (TDD), whereby the sensor can determine (a) the original dose of a drug on the TDD,

(b) the quantity of drug remaining on the TDD, and

(c) The dose which has been delivered to the patient from the TDD over time.

### BACKGROUND OF THE INVENTION

Transdermal delivery systems may employ a medicated device or patch, which may be affixed to an exposed surface of the skin of a patient, thus avoiding the need and the pain associated with drug injections and intravenous drug administration. Transdermal delivery also avoids gastrointestinal metabolism of administered drugs, reducing the elimination of drugs by the liver, and providing a sustained release of the administrated drug. Transdermal delivery may also enhance a patient's compliance with a drug regimen due in part to the relative ease of administration and the sustained release of the medicines.

TDD systems generally rely on pharmaceutical compounds of a molecular weight below 1,000 Daltons. Compounds above 1,000 Daltons in molecular weight may be difficult or impossible to administer transdermally without the aid of electronic, mechanical, or ultrasonic aids.

Passive TDD's have variable delivery rates depending upon a patient's skin structure, placement on the body and the fat content of the patient. Therefore, it is typically difficult to control the dose actually absorbed through the skin of an individual patient. Additionally, current TDD's have no indication of the quantity, or dose, remaining in the patch in a given period of time. There still remains no effective means of gathering dose measurements from TDD's or patch products or determining what dose has been delivered to the patient, or a means of determining the effectiveness of drug delivery from a patch.

Thus, a need exists for a system designed to measure real-time drug delivery through the use of a portable monitor and sensor attached to a transdermal drug delivery device.

According to an aspect of the present invention, a device for measuring in real-time the effectiveness of transdermal drug delivery by the use of an ultrasound sensor communicatively coupled to a control device may be provided. A portable, programmable and ultrasonic sensor, which may be placed directly in contact with a transdermal delivery device or patch for the purpose of sensing, and controlling the delivery of medications contained within the patch. The sensor may be placed directly within a drug-containing TDD or may, alternatively, be worn over a transdermal patch, and may be held in place by adhesives, body affixing straps or other suitable materials and/or devices.

A TDD may contain, for example, a particular medication(s) for treatment of disease or relief of pain. The sensor, when activated may, by its internal timing circuitry, generates an ultrasonic vibration or sonic transmission through the TDD, causing an echo pattern, which may be received by a transducer receiver. The electronic character of the echo pattern may measure the starting dosage amount within the TDD, and later compare that starting value to later values as the medicant is liberated from the TDD over time. Likewise, the sensor, when activated by internal timing circuitry, may generate an ultrasonic vibration or sonic transmission through the skin of a patient, causing an echo pattern, which may be received by a transducer receiver. The electronic character of this echo pattern may measure the dose which actually permeates the skin, and may later be compared to the electronic character signature starting value within the TDD to the later received values as the medicant is liberated from the TDD over time to calculate the quantity of medicant actually received by the patient at any particular point in time.

According to an aspect of the present invention, a sensor may be attached to a TDD or transdermal patch, which may enable the measurement of real time drug delivery through the TDD or transdermal patch into a patient's skin as the active substance within the TDD is deposited or absorbed into the skin of the patient.

According to an aspect of the present invention, a sensor may be attached to a TDD, which may enable the measurement of the quantity of an active substance stored within a TDD, for the purpose of determining the quantity which has been delivered from the TDD and also functioning as a “fuel gauge” for determining the remaining quantity of the active within the, and therefore the remaining “life” of the TDD.

According to an aspect of the present invention, a cymbal type transducer or transducer array (flextensional class V) for my be used as an ultrasonic sensor device to deliver either low or high frequency ultrasound transmissions through the TDD for the purpose of functioning as a “fuel gauge” indicator of the drug remaining within the TDD at any point in time.

According to an aspect of the present invention, a cymbal type transducer or transducer array (flextensional class V) may be used in the sensor device to deliver either low or high frequency ultrasound transmissions through the patients skin to measure the amount of the active substance actually delivered through the patients skin structure, in real-time, as the active substance is being delivered by the TDD or absorbed from the TDD by the skin of the patient, for the purpose of providing data to a control device which will determine and record the actual drug quantity, timing and other related factors to a drug delivery regimen for that individual patient.

The present invention may also provide a control device or monitor which may enable: the measurement of the amount of the active substance delivery through the patients skin structure, in real time, as the active substance is being delivered by a TDD or transdermal patch, or absorbed from the TDD by the skin of the patient; measure and record the amount of the active substance remaining within the TDD for the purpose of functioning as a fuel gauge; for measuring and recording the amount of the active substance remaining which was originally stored within the TDD and which has now been deposited or liberated from the TDD for the purpose of functioning as a fuel gauge in a control function; and, determining and recording the actual drug quantity, timing, control and other related factors related to a drug delivery device.

According to an aspect of the present invention, a rechargeable battery may be located in the device or strap, and may be lightweight and thin and capable of providing suitable power to the at least one sensor and at least one transducer array.

According to an aspect of the present invention, at least one ultrasonic based dosage sensor may operate by measuring a density change of the skin as it is scanned by an ultrasonic signal emitted from the sensor, by relying upon the application of various ultrasound frequencies, intensities and/or phase modulations to generate a sonic pulse and echo wave, which when received by the sensor may enable the accurate measurement of a dose delivered through the skin of the patient or the dosage remaining within the TDD. In particular, acoustical energy may be delivered by a portable, self-powered, programmable ultrasonic transducer placed over a medicament containing TDD allowing for a measured dose within the patch to cross the skin barrier, as part of a dose control system.

According to an aspect of the present invention, at least one sensor including a transducer or array of transducers may be built into the TDD or may be connected to the TDD by any appropriate means.

According to an aspect of the present invention, an ultrasonic phase modulation and alternating waveforms and frequency modulation may be used to achieve the transdermal sensor measurement functions as mentioned above.

According to an aspect of the present invention, a combination of ultrasound with ionophoresis, electroporation, depilatories, or with chemical enhancers may be used to facilitate the transdermal sensor measurement functions as mentioned above.

According to an aspect of the present invention, the sensing capability for each drug or substance, which may be delivered transdermally, may be optimized or customized to enable dose measurement, dose control and to record the dose actually delivered to the patient from a TDD worn on the skin of the patient. The molecular structure of each drug or active substance is different and responds differently to ultrasound. Varying the frequency, intensity, waveform and timing of a sonic or ultrasonic transmission may optimize the measurement of each drug compound stored within a TDD providing crucial information of dose measurement and control including, but not limited to: the quantity of the drug actually absorbed into the skin transdermally, to dose delivered at a particular date, and time; the quantity of the drug liberated from the transdermal drug delivery device or transdermal patch worn on the skin of the patient; and the quantity of the drug remaining within the transdermal drug delivery device or transdermal patch.

According to an aspect of the present invention, use of a dose sensor of this invention may serve to improve the quality of life for patients with diseases or conditions which require periodic administration of drugs by permitting the patients to continually and accurately measure the dose actually delivered to the patient over time. Such sensors can be connected to control devices for the accurate administration of medicinal doses and the recording of the doses actually delivered to the patient. With such dose control and monitoring a more appropriate and customized medication regimen can be established for that individual patient. In addition, the cognitively impaired, elderly, and very young may receive medication with much less supervision, while being absolutely certain of the dose they actually received.

Referring now to FIG. 1, there is shown an illustration of an embodiment of the present invention as it is placed upon the arm of a patient. According to an aspect of the present invention, an enhanced TDD system may comprise a control device 1, which may be, placed directly over a TDD 2. The control device 1, may include an ultrasound sensor. The control device 1 and patch 2 may be attached to the exterior of the patient's skin 3 by means of a strap 4, which may hold the control device 1 and TDD 2 in place. Power for the control device 1 may be provided by a power supply, which may be rechargeable, and may be located within the strap 4. Alternatively, a power supply may be contained within the control device 1 or provided by an external source.

According to an aspect of the present invention, the TDD system may be located on the arm of the patient, placed over the patient's chest, as in the case of nitroglycerin drug delivery, for example, or placed in any other more effective part of the patient's body as determined by the medical personnel administrating the TDD system.

### BRIEF DESCRIPTION OF THE DRAWINGS

Understanding of the present invention will be facilitated by consideration of the following detailed description of the preferred embodiments of the present invention taken in conjunction with the accompanying drawings, in which numerals refer to like parts, and wherein:

FIG. 1 is an illustration of the design of a conventional Matrix type transdermal patch illustrating a drug reservoir which is placed between two membrane films.

FIG. 2, is an illustration of the design of a conventional Reservoir type transdermal patch illustrating a drug reservoir which is admixed with adhesive, and then placed between two membrane films.

FIG. 3 is an illustration of the structure of human skin;

FIG. 4 is a Photograph showing a typical transdermal patch which has been placed upon the arm of a patient.

FIG. 5 illustrates an embodiment of the present invention; which is a sensor for measuring the dose present upon a transdermal delivery device or patch.

FIG. 6 is a reflective transducer design for directing the ultrasound to a specific target.

FIG. 7 Illustrates the use of an alternating waveform, a conversion from sawtooth to square wave, as generated by the frequency driver of the present invention;

FIG. 8 illustrates an embodiment of a transducer of the present invention, and the use of a polymer potting used as a resonance compatible coupling agent coating over the surface of the transducer element;

FIG. 9. Illustrates an array of transducers that may be used in an embodiment to the present invention to enhance sonic efficiency and to provide multiple sensor sites to the skin;

FIG. 10 is an illustration of multiple transducers which may create an echo sensor return pattern for use with an embodiment of the present invention, consisting of up to 9 individual transducer elements;

FIGS. 11A and B illustrate an embodiment of a modified transdermal patch in accordance with the present invention, wherein the sensor slides into the patch.

FIG. 12 Illustrates the sensor placed over top and in contact with a matrix type transdermal patch.

FIG. 13 Illustrates the sensor placed over top and in contact with a reservoir type transdermal patch.

FIG. 14 (bottom view) and FIG. 15 (top view) illustrates an embodiment of a patch cap transdermal delivery device which can be fitted with the present invention.

FIG. 16 (top view) and FIG. 17 (bottom view) illustrates an embodiment of a flexible transdermal patch which can be fitted with the present invention.

FIG. 18 is a photograph of the device described in FIGS. 16 and 17 as applied to the upper left arm and to the right side abdomen of a patient.

FIG. 19 illustrates an embodiment of the present invention as worn by a patient; on the arm;

FIG. 20 illustrates an embodiment of the present invention as worn by a patient; on the abdomen;

FIG. 21 illustrates the design of a control device which can indicate the dose remaining upon a transdermal delivery device or patch, in real times as the sensor feeds accurate and timely data on the performance of the TDD.

FIG. 22 is a photograph of the Franz cell used in Experiment 1.

### DETAILED DESCRIPTION OF THE INVENTION

It is to be understood that the figures and descriptions of the present invention have been simplified to illustrate elements that are relevant for a clear understanding of the present invention, while eliminating, for purposes of clarity, many other elements found in a typical drug delivery devices. Those of ordinary skill in the art will recognize that other elements are desirable and/or required in order to implement the present invention. However, because such elements are well known in the art, and because they do not facilitate a better understanding of the present invention, a discussion of such elements is not provided herein. The disclosure hereinbelow is directed to all such variations and modifications to these technologies known, and as will be apparent, to those skilled in the art.

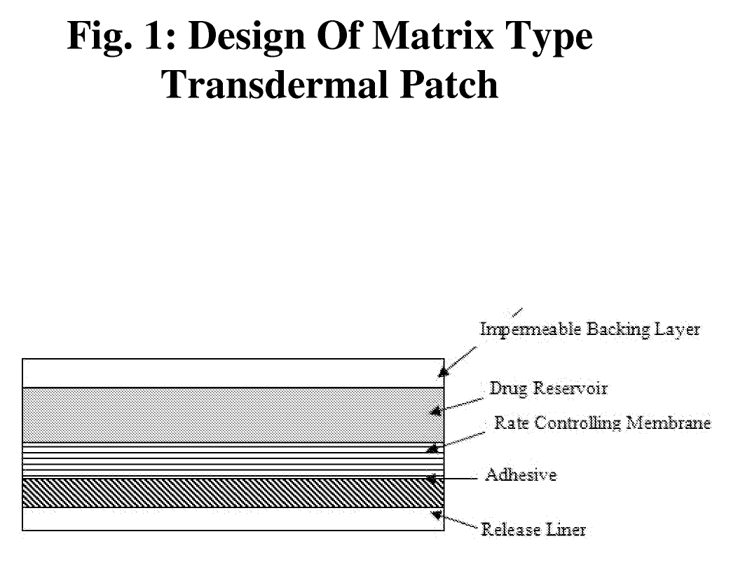

FIG. 1 is an illustration of the design of a conventional Matrix type transdermal patch illustrating a drug reservoir which is placed between two membrane films.

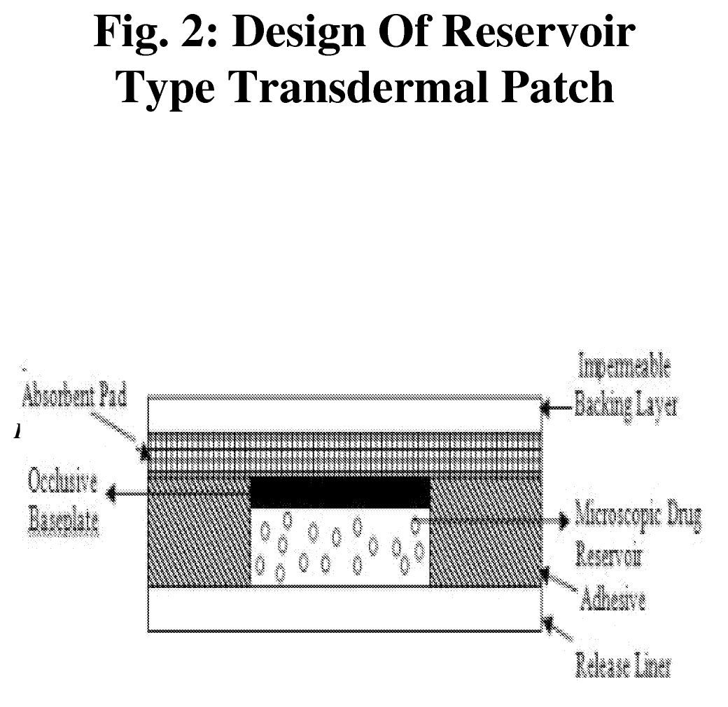

FIG. 2, is an illustration of the design of a conventional Reservoir type transdermal patch illustrating a drug reservoir which is admixed with adhesive, and then placed between two membrane films.

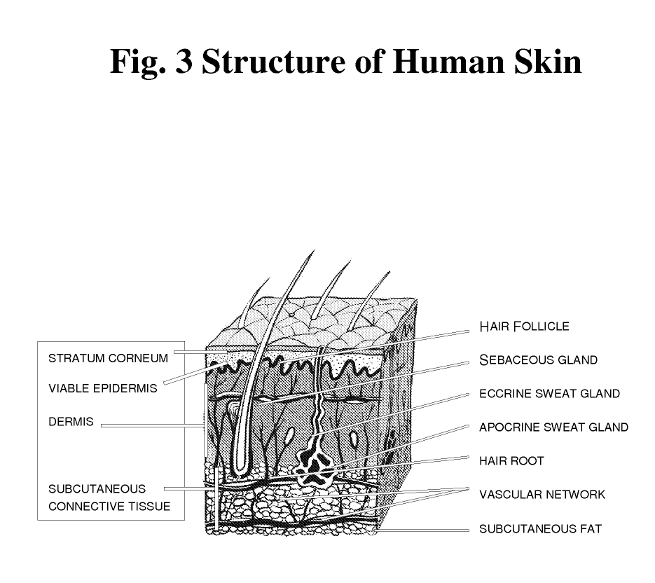

FIG. 3 is an illustration of the structure of human skin;

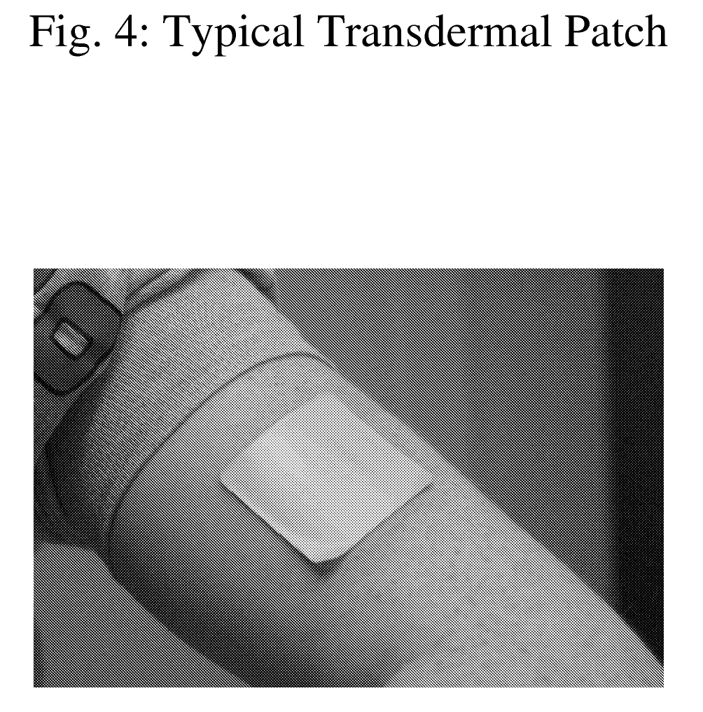

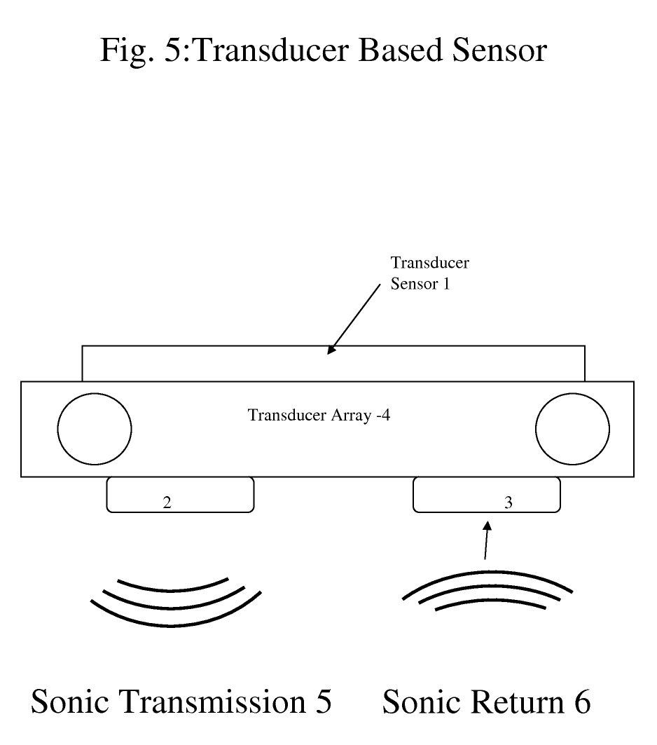

FIG. 4 is a Photograph showing a typical transdermal patch which has been placed upon the arm of a patient.

Referring now to FIG. 5, there is shown an illustration of an embodiment of the present invention which involves a sensor device (5.1) composed of an ultrasonic transducer array (5.4) which directs an ultrasonic transmission (5.5) from a transmitter element (5.2), via a transmitting transducer (5.2) to a transdermal delivery device (TDD). An echo return pattern (5.6) is received by a receiver transducer (5.3) and can relay that data to a control device and indicator which will measure the quantity of liquid present within the TDD as measured in milliliters (ml).

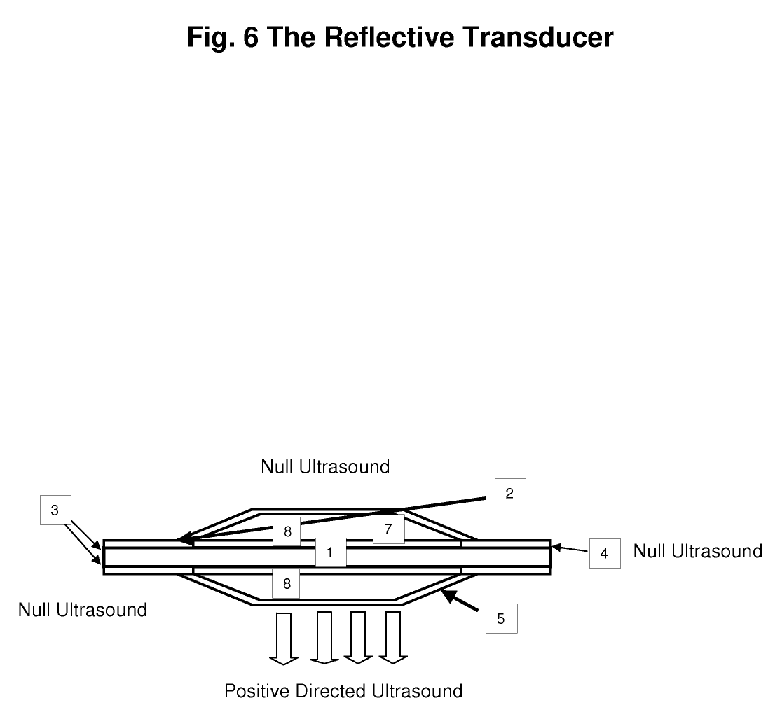

FIG. 6 is a reflective transducer design for directing the ultrasound to a specific target. A piezoelectric crystal

FIG. 6 illustrates the design of an ultrasonic transducer (6.1), which is the preferred embodiment of the transducer element of this invention. From FIG. 6 it can be seen that a cymbal transducer (6.1) is based upon a piezoelectric disc (6.1) such as PZT4 (Piezokinetics Corp. Bellefonte, Pa.), connected to two metal caps (6.2 and 6.5) composed of titanium foil preferably.

FIG. 6 illustrates that there is a hollow air space (6.8) between the piezoelectric disc (6.1 and the end caps (6.2 and 6.5). The end caps (6.2 and 6.5) are connected to the piezoelectric disc (6.1) by a non-electrically conductive adhesive (6.3 and 6.4) to form a bonded layered construction to the transducer (6.1). The upper cap (6.2) is coated on the interior with an epoxy film layer (6.7), which will retard harmonic vibration of the top cover (6.2) and therefore direct ultrasound only towards the bottom of the transducer device.

This transducer design offers a thin, compact structure more suited for a portable ultrasonic drug delivery apparatus. Additionally this transducer offers greater efficiency for the conversion of electric power to acoustically radiated power. Applicant chose this design of a transducer also because of its potential to be battery powered and its small, lightweight features.

This transducer element is both suitable for transmitting and receiving ultrasound.

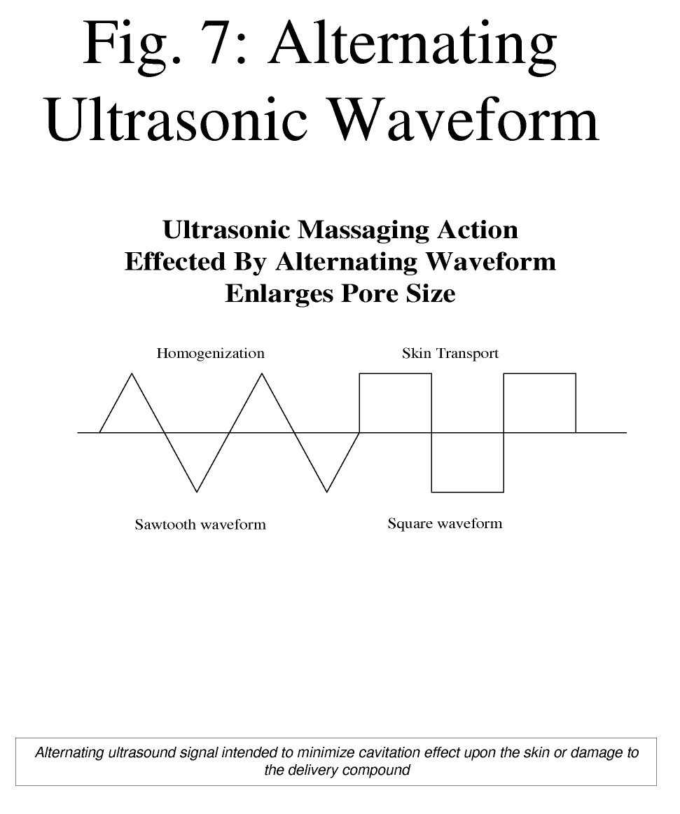

FIG. 7 illustrates the use of an alternating waveform, a conversion from sawtooth to square wave, as generated by the frequency driver of the present invention. The transducer design illustrated in FIG. 6 is capable of delivering multiple ultrasonic waveforms and frequency levels. The use of an ultrasonic signal which converts from one waveform to another has been found to minimize cavitation heating effects upon whatever surface the ultrasound is transmitted through.;

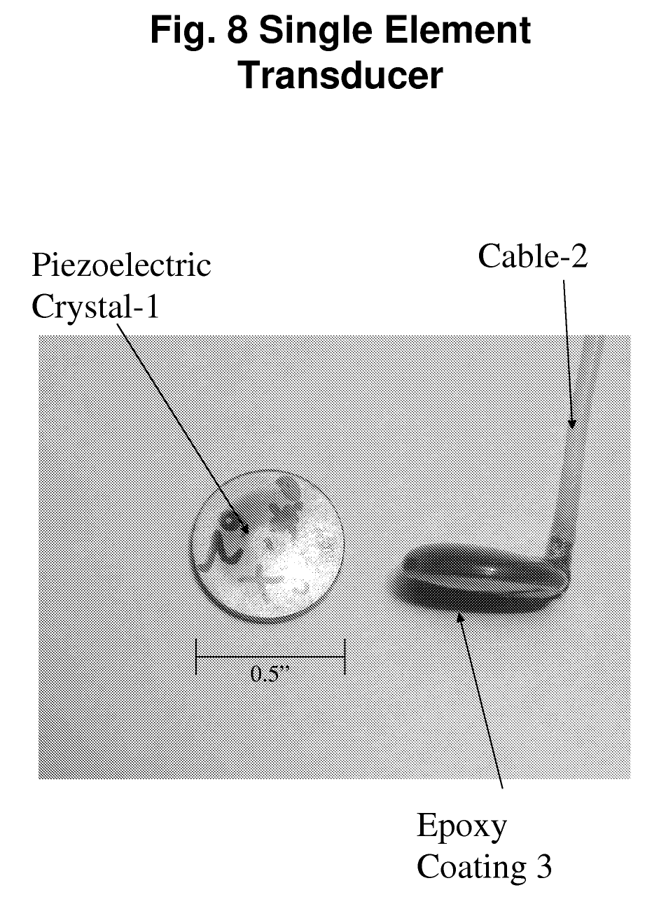

FIG. 8 illustrates an embodiment of a transducer of the present invention, which involves a transducer crystal as described in FIG. 6 (8.1) which itself is coated by a polymer potting (8.3) used as a resonance compatible coupling agent coating over the surface of the transducer element (8.1) and is electrically connected by a cable (8.2).

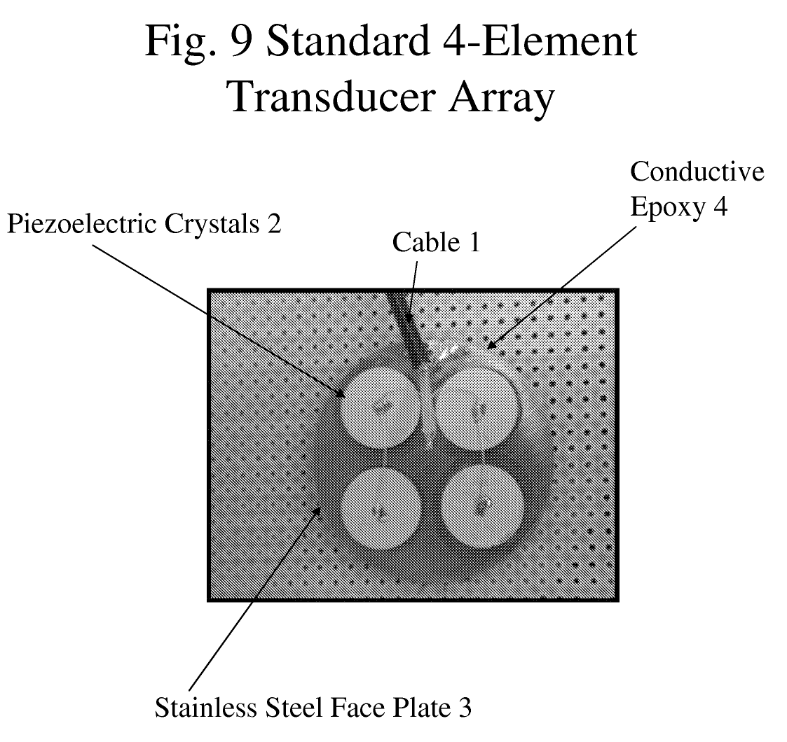

FIG. 9. Illustrates an array of transducers that may be used in an embodiment to the present invention to enhance sonic efficiency and to provide multiple sensor sites to the skin. In this configuration two transducer elements (9.2) are used to transmit ultrasound while two are used to receive the echo ultrasonic transmission returning form interaction with a transdermal delivery device or patch. The four transducers are fitted onto a stainless steel face plate (9.3) and connected by an electric cable (9.1). A conductive epoxy (9.4) is used to secure the transducer discs (9.2) onto the face plate's surface (9.3).

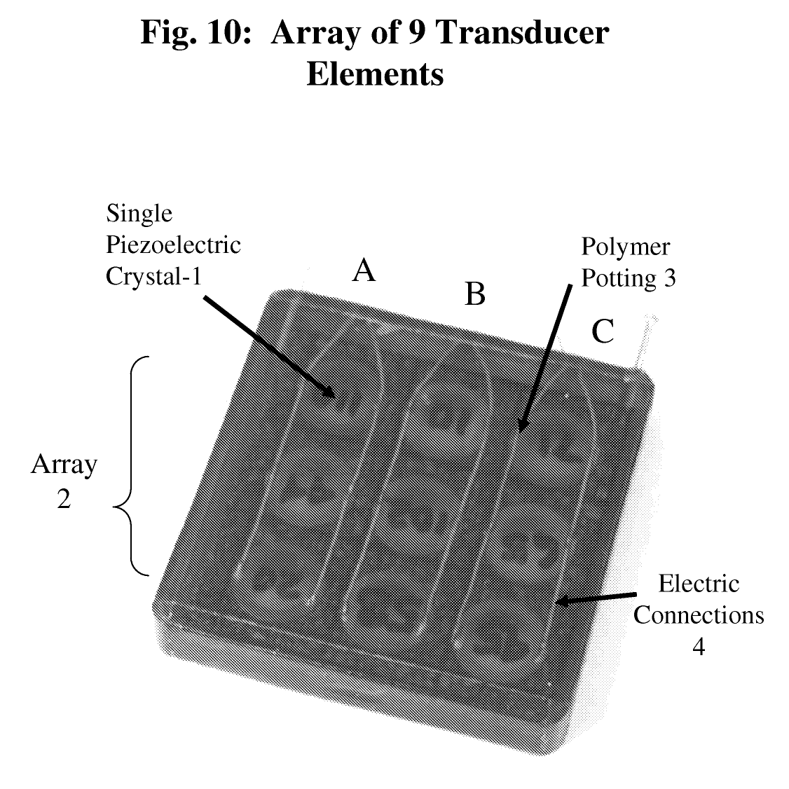

FIG. 10 is an illustration of multiple transducers which may create an echo sensor return pattern for use with an embodiment of the present invention, consisting of up to 9 individual transducer elements. In this configuration there are 3 rows of transducers consisting of 3 discs per row. (9.1). the transducer row A and C are transmitting transducers. The middle Row of B transducers are receiving transducers.

The transducer elements in the A and C rows are used to transmit ultrasound while the elements in the middle B row are used to receive the echo ultrasonic transmission returning form interaction with a transdermal delivery device or patch. The nine transducers are fitted onto a polymer potting board (9.3) and connected by an electric cable (9.4). The transducer array (9.2) is held in place by the polymer potting material (9.3).

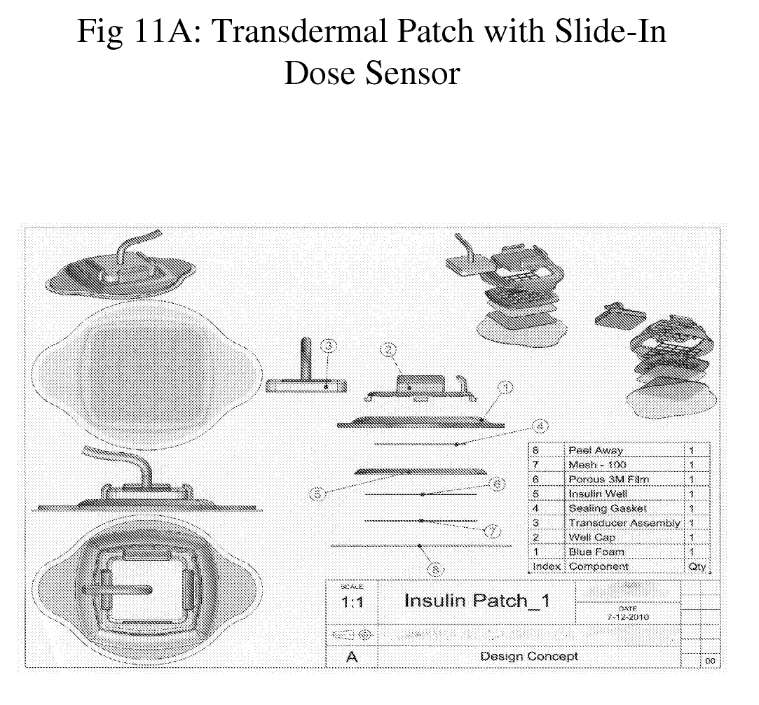

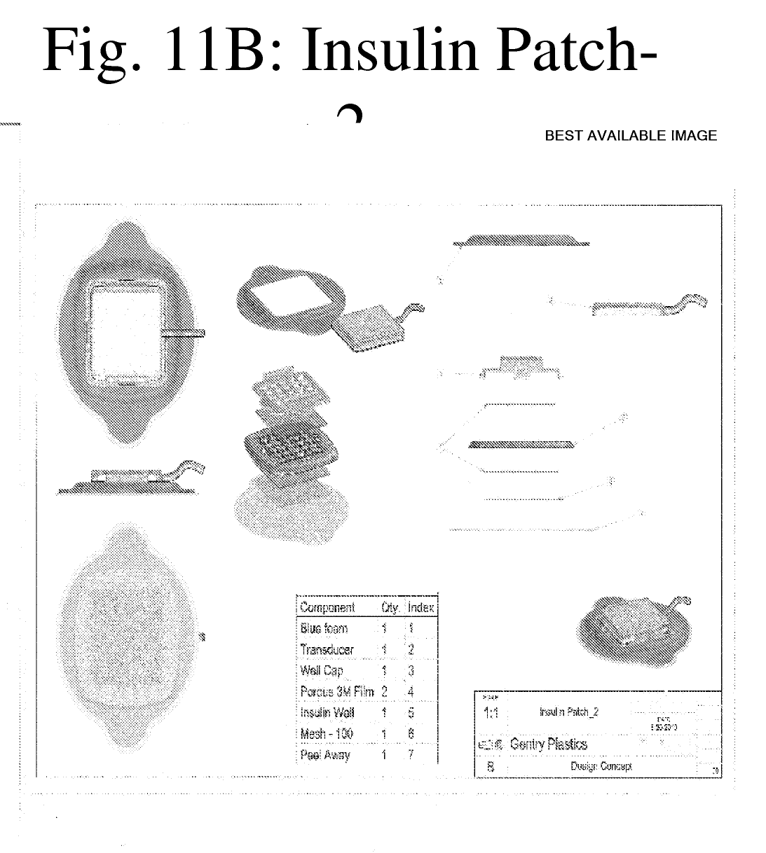

FIGS. 11A and B illustrate an embodiment of a modified transdermal patch in accordance with the present invention, wherein the sensor slides into the patch.

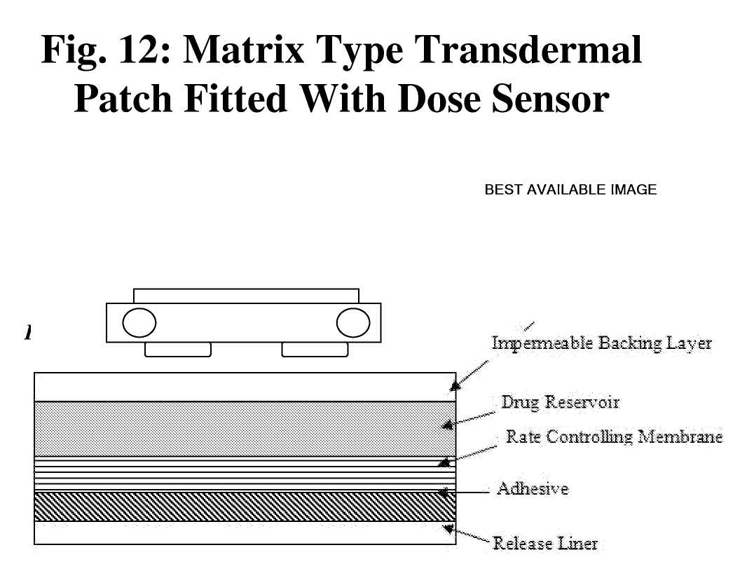

FIG. 12 Illustrates the sensor placed over top and in contact with a matrix type transdermal patch.

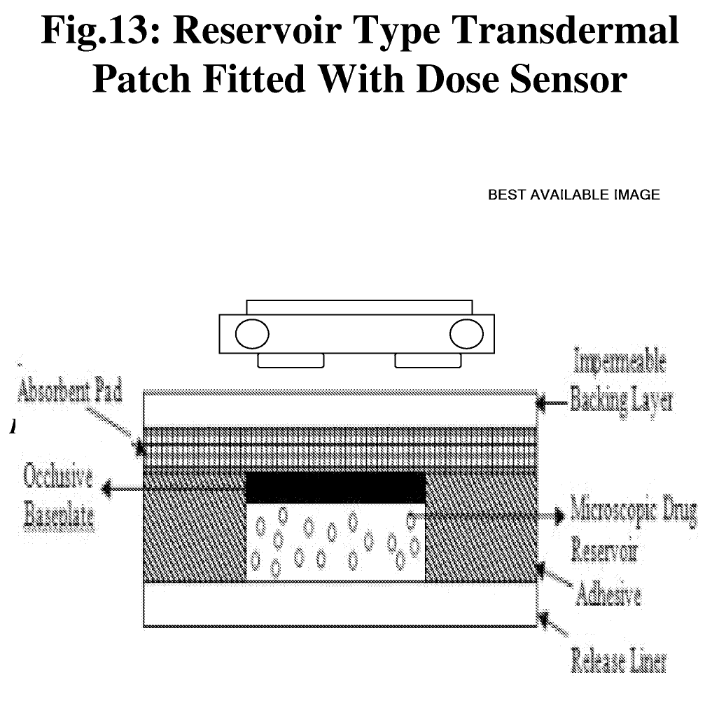

FIG. 13 Illustrates the sensor placed over top and in contact with a reservoir type transdermal patch.

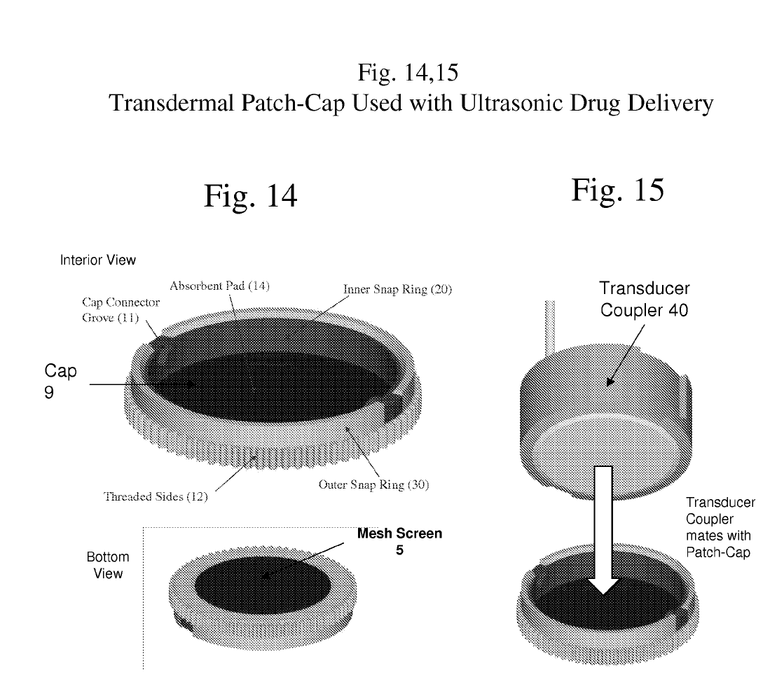

FIG. 14 (bottom view) and FIG. 15 (top view) illustrate an embodiment of a patch cap transdermal delivery device which can be fitted with the present invention.

### Patch-Cap Design Transdermal Delivery Device

FIGS. 14 and 15 illustrates an Active transdermal delivery device termed a Patch-Cap, designed to mate with a transducer coupler and sensor for the purpose of delivering insulin, from the PATCH-Cap and sensing the dose remaining upon the absorbent [pad portion of the TDD.

Conventional TDD's are flexible patches. The Patch-Cap is an example of a hard form of TDD. It uses an absorbent pad (14.14) to hold the drug.

FIG. 15 illustrates how the Transducer coupler is mated to the Patch-Cap illustrated in FIG. 14.

FIG. 14 is an illustration of a Transdermal Patch Cap designed to deliver insulin transdermally using ultrasonic propagation. The Patch Cap consists of an absorbent pad (14.14), which is placed within a holder, the outer snap ring (14.30). It is locked into place by an inner snap ring (14.20) and then is used to absorb a drug, particularly insulin. The Cap (14.9) has threaded sides (14.12) in the preferred design and a cap connector grove (14.11) which fits into an ultrasonic emission transducer coupler (15.40). A mesh screen (14.5) is placed across the absorbent pad (14.14) at the bottom of the patch cap. Using this construction a transdermal patch-cap forms minute droplets of the drug upon the skin's surface either through passive or active means. This design patch is especially suited for ultrasonic drug delivery.

FIG. 15 illustrates how an ultrasonic transducer coupler and sensor (15.40) is mated to the Patch-Cap illustrated in FIG. 14, when used with ultrasonically based drug delivery systems.

### Active Transdermal Delivery Device which Incorporates a Two-Part Design, Incorporating a Transducer Coupler which is Slid into or Snaps onto a Flexible Patch

FIG. 11A is an Active transdermal delivery device which incorporates a two-part design, incorporating a transducer coupler which is slid into or snaps onto a flexible patch, fitted with multiple absorbent pads and a mesh screen for the purpose of delivering insulin.

FIG. 11B is an another version of an Active transdermal delivery device which incorporates a two-part design, incorporating a transducer coupler which is slid into or snaps onto a flexible patch, fitted with multiple absorbent pads and a mesh screen for the purpose of delivering insulin.

A Backbone layer (11.1) is the base of the patch. A transducer assembly (11.3) snaps onto the patch 11.1) at the top of the patch by connecting to the well cap (11.2) Directly at the top of the patch a film which may allow ultrasound to penetrate it (11.6) is placed directly above the Absorbent Well (11.5), which contains at least two absorbent pads, on top of the other, into which a dose of a particular substance or drug may be stored. In the initial application of this design insulin is stored within each absorbent pad so that the patch may be used to treat diabetes. A sealing gasket (11.4) is placed around the well (11.5) to isolate it from any adhesive used in the border of the patch (11.1). A mesh screen (11.7) is installed at the bottom of the patch ahead of a peel away film (11.8).

### Transdermal Delivery Device Incorporating an Alternative Flexible Patch Design with Snap-Connector to a Transducer Coupler Capability

FIG. 66 is a Top View depiction of a flexible transdermal patch design modified to use the mesh screen at the bottom of the batch. This particular design uses an absorbent pad to hold the drug and the drug is liberated under an Active control fashion using ultrasound.

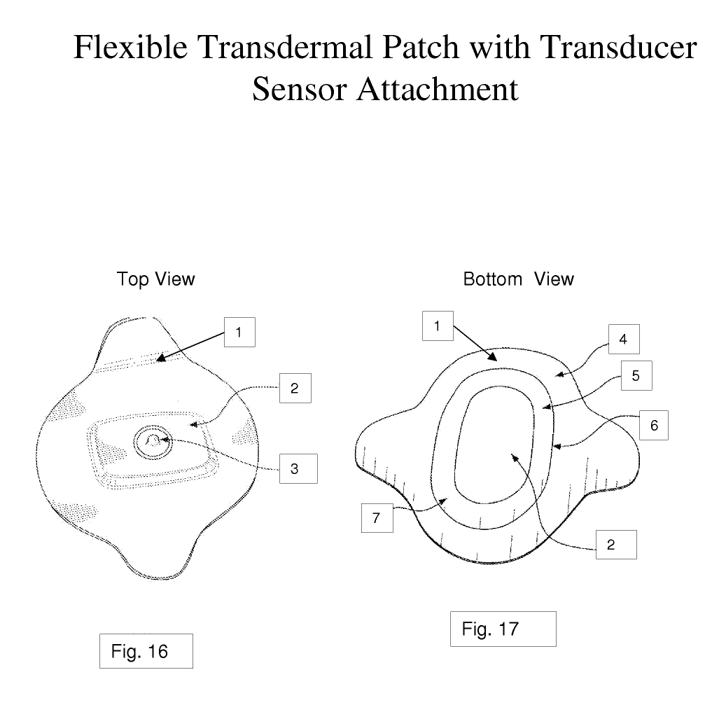

FIG. 16 is a Top View depiction of a flexible transdermal patch design modified to use the mesh screen at the bottom of the batch. This particular design uses an absorbent pad to hold the drug and the drug is liberated under an Active control fashion using ultrasound. In this design the flexible patch may be used passively with low molecular weight drugs, generally below 1,000 Daltons. On the top of the patch (16.1) is a snap (16.3) which can attach the patch (16.1) to an ultrasonic transducer device, which in turn sends an ultrasonic transmission through the patch and liberates the drug stored within the absorbent pad section (16.2) onto the surface of the skin.

In FIG. 17, the rear side of the flexible patch is shown. The backbone (17.1) or border of the patch which comes into contact with the skin has an adhesive border (17.4) to stick the patch to the skin's surface. The adhesive (17.4) does not come into contact with the drug directly. The absorbent pad or stacked pads (7.2) is placed within a bordered well or reservoir section (17.5) which isolates it from contact with the adhesive layer (117.4) through the use of a gasket (17.6). At the very bottom of the patch a mesh screen (7.7) is placed across the drug reservoir (17.5) and over the absorbent pad or collection of stacked absorbent pads (17.2). Using this construction a transdermal patch forms minute droplets of the drug upon the skin's surface, either through passive or active means. This design patch is especially suited for ultrasonic drug delivery.

In this design a transducer sensor (17.3) can be installed directly within the patch which will serve to transmit ultrasound and to receive the echo transmission and which will determine the dose remaining on the patch.

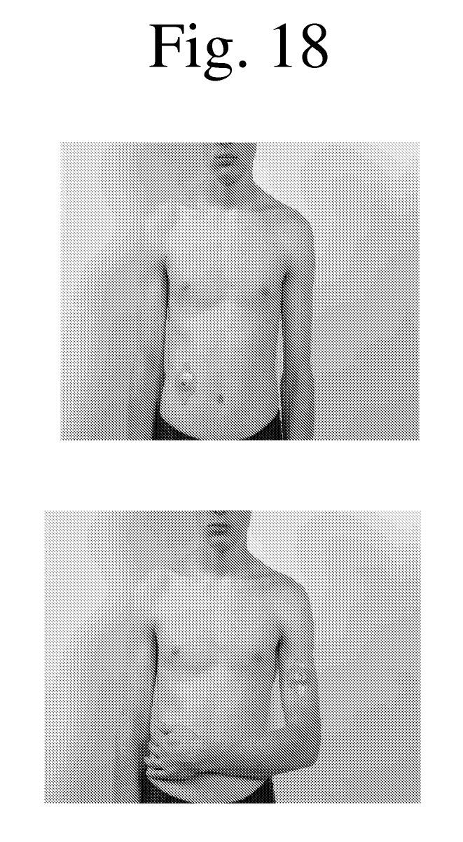

FIG. 18 is a photograph of the device described in FIGS. 16 and 17 as applied to the upper left arm and to the right side abdomen of a patient.

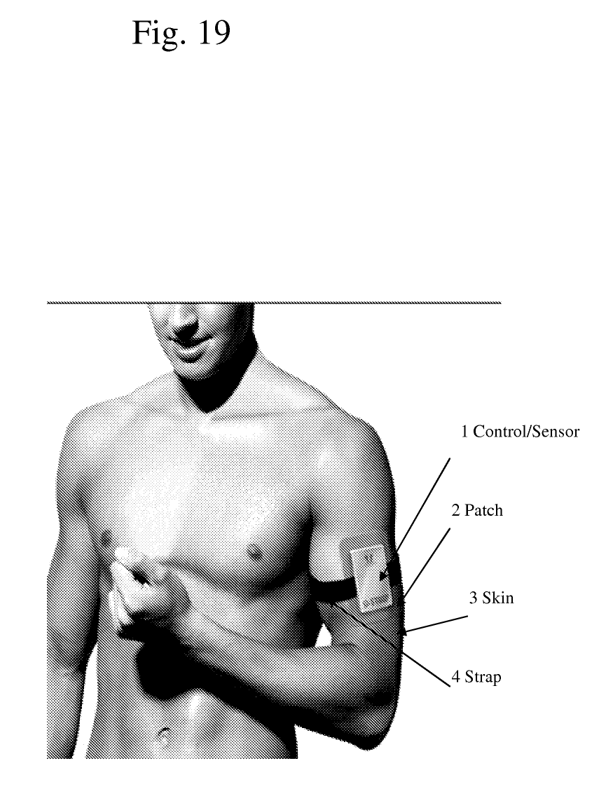

FIG. 19 illustrates an embodiment of the present invention as worn by a patient; on the arm. In this system the control device (19.1) interprets the readings form the patch sensor (19.2) and illustrates the dose originally upon the patch, the dose remaining and calculates the dose delivered to the patient. The patch and the accompanying sensor (19.2) is attached to the skin (19.3) by way of a strap (19.4) or an adhesive layer.

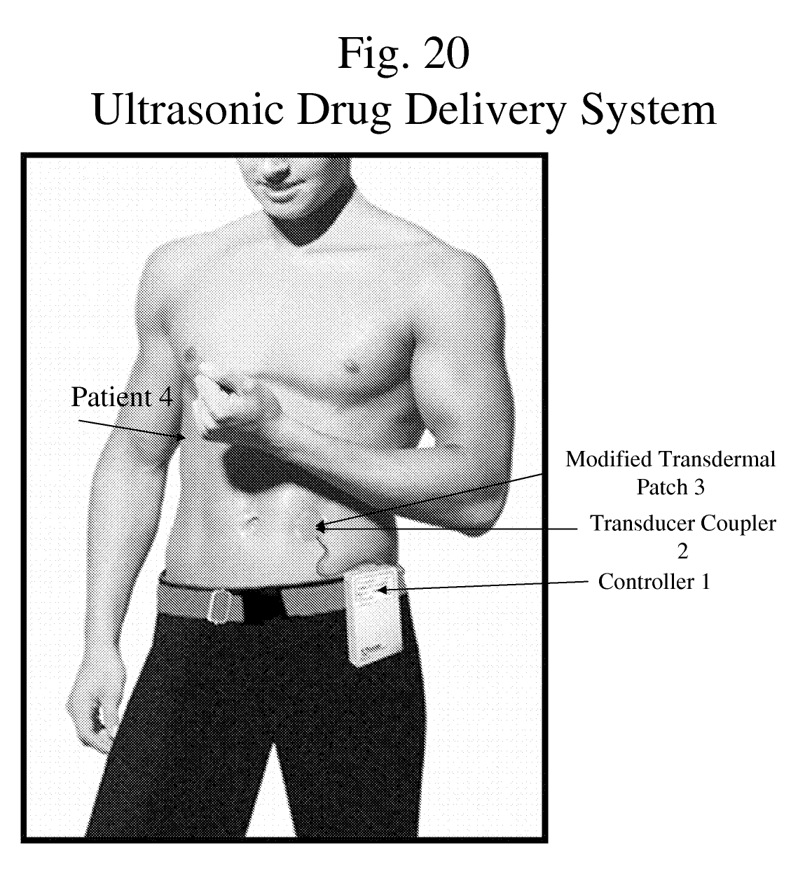

FIG. 20 illustrates an embodiment of the present invention as worn by a patient; on the abdomen. In this system the control device (20.1) interprets the readings form the patch sensor (20.2) and illustrates the dose originally upon the patch (20.3), the dose remaining and calculates the dose delivered to the patient. The patch and the accompanying sensor (20.2 and 3) is attached to the skin (20.4) by way of an adhesive layer.

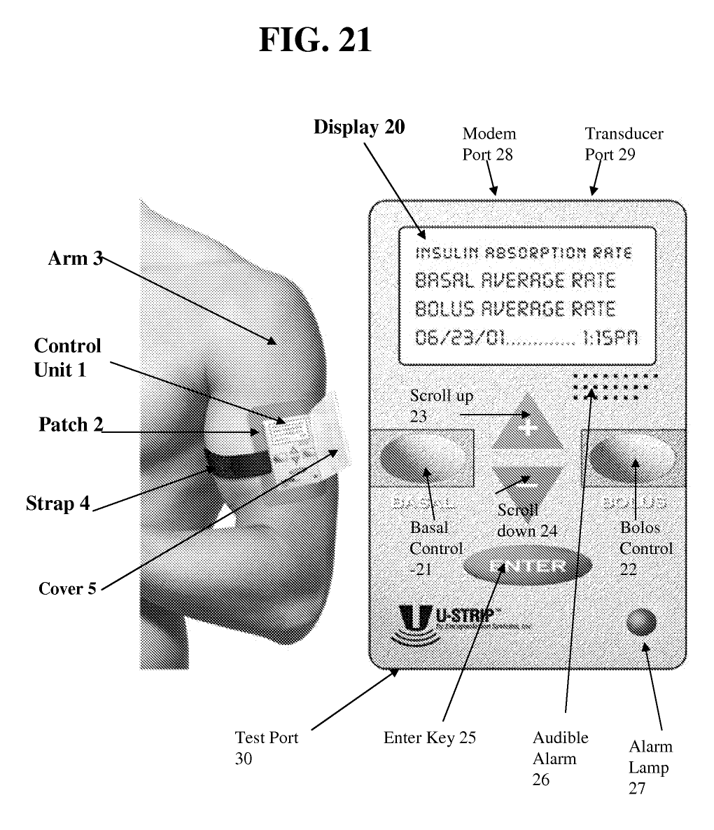

FIG. 21 illustrates the design of a control device which can indicate the dose remaining upon a transdermal delivery device or patch, in real time, as the sensor feeds accurate and timely data on the performance of the TDD. FIG. 21 is depicted on the arm of a patient, wherein the cover (21.5) of the control device (20.1) is in an open position. Further, FIG. 21 illustrates an enlarged view of the control device (20.1), which may include, but is not limited to, a monitor, a sensor control, a power supply, modem, transducer, transducer array, and a processor. The control device (20.1) may further include: a display (20.20), a basal control button (20.21), a bolos control button (20.22), a scroll up button (20.23), a scroll down button (20.24), an entry key (20.25), an audible alarm (20.26), an alarm lamp (20.27), a modem port (20.28), a transducer port (20.29), and a test port (20.30).

Referring now to FIG. 7, there is shown an alternating sonic waveform that may be produced by the present invention that may enhance the capability of the control device 1 of detecting the dose amount remaining within a TDD or delivered through the skin of a patient, wherein a combination of a sawtooth and a square waveform signal may be efficient at dose sensing when the echo return of a sonic transmission through a TDD is examined by the control device 1.

The transducers are designed with 4 transducer elements within an array. All 4 transducers transmit a Driving force of 20-30 kHz ultrasonic frequency at 125 mW/sq. cm intensity using an alternating ultrasonic waveform consisting of 100 milliseconds on saw tooth waveform and then 100 milliseconds on square waveform before converting back to sawtooth. It is believed the sawtooth waveform component enlarges the skin pores and the square waveform drives the drug from the patch through the skin. In the case of the sensor two of the transducers in the array then convert to a higher frequency transmission every 60 seconds. That frequency, 80 kHz, at the same intensity, 125 mW/sq. cm, sends an ultrasonic pulse through the absorbent pad of the patch which lasts for only 100 milliseconds using a sinusoidal waveform. That pulse is like a sonar transmission and has both a forward transmission and a return transmission or echo. That echo is received by the other two transducers in the array and produces a voltage, which corresponds to the degree of wetness of the liquid content on the absorbent pad.

The same transducer array may be used to push the drug from the patch and deliver the drug transdermally through the patient's skin, and to measure the dose. On the driving setting the alternating waveform and 20-30 kHz frequency is used. On the drug sensing mode the frequency converts to sine wave and jumps to 80 kHz. The echo voltage in the receiving transducers gives a measure of the quantity of the liquid remaining within the absorbent pad portion of the patch. Refer to following experiment:

### Experiment-1

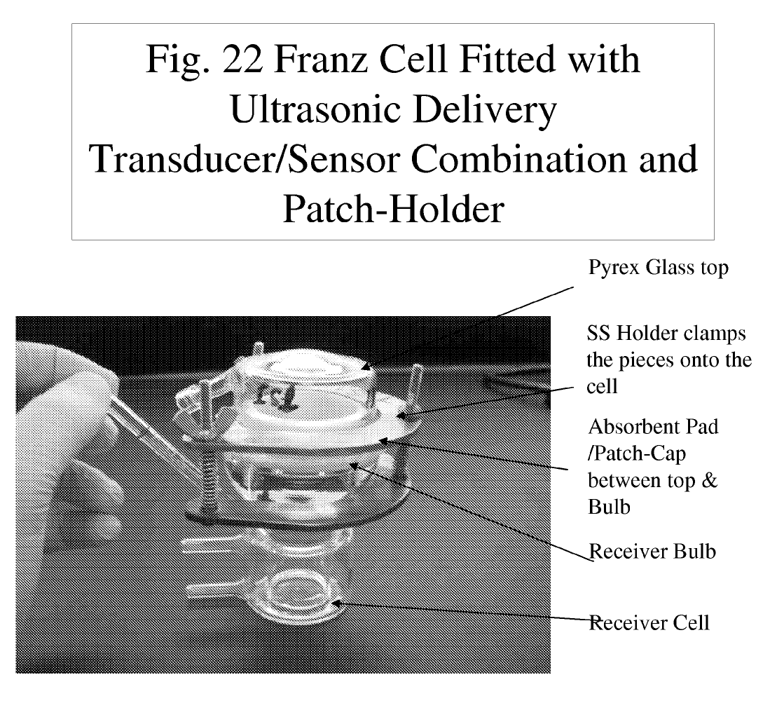

1. A transdermal patch holder as shown in FIG. 14 and FIG. 15 is constructed according to the following dimensions: 2.73 in. Diameter×0.63 in. Height.
2. An absorbent pad composed of cellulose material, model no. Vicell 9009, supplied by Buckeye Products Company, with a thickness of 0.92 mm/ply is shaped into a circular pad with a diameter of 1.6 in.
3. The absorbent pad is then loaded with 1.0 ml of Humulin Reg. Insulin supplied by Eli Lilly using a hypodermic. This constitutes 100 units of insulin upon the absorbent pad. The pad is then sealed with saran film on both faces and the wetted pad is then placed into the Patch Cap holder.
4. A 4-element transducer array, Model no BKR-1007-37, is mated against the insulin loaded absorbent pad in the holder, constructed as seen in FIG. 15. This transducer array has the ability to transmit two different ultrasonic transmissions: (A) Driving force of 20-30 kHz ultrasonic frequency at 125 mW/sq. cm intensity using an alternating ultrasonic waveform consisting of 100 milliseconds on sawtooth waveform and then 100 milliseconds on square waveform before converting back to sawtooth and (B) 80 kHz, at 125 mW/sq. cm, sinusoidal waveform for only 100 milliseconds and timed to send a pulse every 60 seconds through the absorbent pad. Control of this function is managed by an ultrasonic driver circuit Model No. ESI-25 B-1, provided by Transdermal Specialties
5. Two of the transducers in the array are attached to an oscilloscope to measure the peak-to-peak voltage of the measured echo pattern when the transducers are at setting B.
6. The absorbent pad was measured with the insulin loaded as a staring point and various weight loss measurements were made over time as the ultrasound at setting (A) drove the insulin from the absorbent pad, fitted onto a Franz Diffusion cell as seen in FIG. 22. These weight measurements were compared to the peak-to-peak voltage over the echo transducers to develop a voltage match to the liquid content remaining in the pad at that time.
7. The experiment was repeated a total of 5 times and the average readings adjusted.
8. Next a check was made for the echo voltage to ascertain whether it corresponded to a certain liquid content upon the absorbent pad portion of the Patch-Cap.

### Results

Starting weight of pad un-loaded: 0.0995 grams
Weight of liquid insulin starting at 100 units: 1.00 grams (Lispro Insulin U-100 strength known commercially as Humalog® from Lilly Co.
Weight of pad with 1.00 ml of insulin, equivalent to 100 units of insulin: 1.09995 grams
Starting voltage of Echo Pulse through fully loaded pad: 222 milli volts

| Run No | 1 | 2 | 3 | 4 | 5 |
|---|---|---|---|---|---|
| Starting absorbent pad weight with 100 units of | 1.0999 | 1.0997 | 1.0998 | 1.0992 | 1.0994 |
| Lispro insulin loaded | | | | | |
| Ultrasonic alternating sawtooth waveform timing | 100 | 100 | 100 | 100 | 100 |
| Ultrasonic square waveform timing in mess | 100 | 100 | 100 | 100 | 100 |
| Ultrasonic frequency of Active Ultrasound | 23 kHz | 23 kHz | 23 kHz | 23 kHz | 23 kHz |
| Sensing ultrasound frequency | 175 kHz | 175 kHz | 175 kHz | 175 kHz | 175 kHz |

| | Pad Wt. | Pad Wt. | Pad Wt. | Pad Wt. | Pad Wt. |
|---|---|---|---|---|---|
| | in loss | in loss | in loss | in loss | in loss |
| | grams | grams | grams | grams | grams |
| Ultrasonic activation | | | | | |
| in minutes | | | | | |
| 0 | 0 | 0 | 0 | 0 | 0 |
| 5 | 0.16 | 0.15 | 0.16 | 0.14 | 0.15 |
| 10 | 0.24 | 0.23 | 0.24 | 0.21 | 0.23 |
| 15 | 0.32 | 0.31 | 0.32 | 0.29 | 0.29 |
| 20 | 0.432 | 0.42 | 0.44 | 0.41 | 0.38 |
| 25 | 0.48 | 0.47 | 0.48 | 0.46 | 0.44 |
| 30 | 0.58 | 0.57 | 0.58 | 0.51 | 0.49 |
| Ultrasonic Sensing Echo | | | | | |
| Voltage, P-P in mV | | | | | |
| 0 | 222 | 222 | 222 | 222 | 222 |
| 5 | 201 | 201 | 201 | 201 | 201 |
| 10 | 175 | 175 | 175 | 175 | 175 |
| 15 | 156 | 156 | 156 | 156 | 156 |
| 20 | 132 | 132 | 132 | 132 | 132 |
| 25 | 129 | 129 | 129 | 129 | 129 |
| 30 | 121 | 121 | 121 | 121 | 121 |

### Echo Voltage Comparable to Dose Remaining Upon Patch

| | Dose | Dose |
|---|---|---|
| | Remaining | delivered |
| Final Ultrasonic Echo Voltage | On Patch in | to Skin in |
| Correlation to Patch Dosing | units | units |
| 222 | 100 | 0 |
| 201 | 84 | 16 |
| 175 | 76 | 24 |
| 156 | 68 | 32 |
| 132 | 56.8 | 43.2 |
| 129 | 52 | 48 |
| 121 | 42 | 58 |

Since 100 units of insulin corresponded to 1 ml or 1 gram of total liquid upon the patch it could easily be calculated the dose remaining upon the patch, and the dose delivered from the patch and the delivery rate over time. A microprocessor fitted into a control device, as depicted in FIG. 21, could easily make the calculations and display the dose record in real time. Additionally such a device could record the dosing history over time and down load a dosing report to a physician for dose tracking.

### SUMMARY

The echo voltage in the sensor varied as the liquid loading, the insulin content, of the absorbent pad decreased. For example at 5 minutes of continuous ultrasound treatment 16% of the starting weight of the insulin had been lost from the pad and the corresponding echo voltage of the sensor had dropped from 222 mV to 201 mV, a decrease of nearly 10%. By minute 20 of continuous ultrasonic exposure the liquid loss was 43.2 units of insulin comparable to an Echo voltage of 132 mV.
The experiment confirmed that a sensor was possible to trigger off of the liquid content within the patch. Insulin is 100 units=1 ml so the liquid content loss over time can be calculated by a microprocessor in the ultrasonic control device to indicate the dose delivered from the patch in relation to the ultrasonic voltage data coming from the sensor arrangement.
This sensor method will determine the dose remaining in the patch at any given time, and therefore can calculate the dose delivered from the patch. It can be inferred that the dose delivered went into the patient to provide a dose controlling mechanism for ultrasonic drug delivery.

An echo sensor may indicate 100 units within a patch at 100% original liquid concentration. As time goes on, for example, it could indicate a 45% reduction in the original solution strengthen which would indicate that 45 units of insulin were delivered from the patch and that 55 units remain.

Several combinations of sonic sensors are possible including: low frequency and low intensity ultrasound; high frequency, high intensity ultrasound; low frequency and high intensity ultrasound; high frequency, low intensity ultrasound, as long is care that the sensor signal does not generate enhanced cavitation within the stratum corneum which could result in skin burning and damage to the drug either within the TDD or as it travels through the skin.

The disclosure herein is directed to the variations and modifications of the elements and methods of the invention disclosed that will be apparent to those skilled in the art in light of the disclosure herein. Thus, it is intended that the present invention covers the modifications and variations of this invention, provided those modifications and variations come within the scope of the appended claims and the equivalents thereof.

I claim:

1. A transdermal substance delivery device, comprising:
At least one ultrasonic transducer for genera rating at least one ultrasonic transmission for inducing movement of at least one substance into a tissue;
Said at least one ultrasonic transmission and at least one sensor positioned with said at least one transducer to sense reflected ultrasound transmissions;
Wherein, said sensed ultrasound transmissions are indicative of substance actually moved into said tissue, for determining the starting dose, the dose liberated from a transdermal delivery device or patch, and the remainder dose still within the transdermal delivery device or patch.

2. The transdermal drug delivery device of claim 1, wherein said ultrasound has a frequency in the range of about 20 KHz to 50 MHz.

3. The transdermal drug delivery device of claim 1, wherein said ultrasound has an intensity of about 125-mW/sq. cm to 3.0 W/sq. cm.

4. The transdermal drug delivery device of claim 1, wherein said ultrasound utilizes an alternating waveform to avoid cavitation heating within the drug or within the tissue.

5. The transdermal drug delivery device of claim 4, wherein said alternating waveform comprises a sawtooth waveform.

6. The transdermal drug delivery device of claim 4, wherein said alternating waveform comprises a square waveform.

7. The transdermal drug delivery device of claim 1, wherein said ultrasound is applied substantially continuously.

8. The transdermal drug delivery device of claim 1, wherein said ultrasound is pulsed.

9. The transdermal drug delivery device of claim 1, further comprising a control device for managing the ultrasonic transmissions, and receiving ultrasonic signals which can be calculated to determine the dose remaining upon a transdermal delivery device or patch, indicating that data to the patch wearer or also recording such data for transmission to a trained medical professional for analysis later.

10. A transdermal substance delivery device, which can be fitted over or connected to a transdermal delivery device or patch, comprising at least one ultrasonic transducer for genera rating at least one ultrasonic transmission for inducing movement of at least one substance into a tissue; Said at least one ultrasonic transmission and at least one sensor positioned with said at least one transducer to sense reflected ultrasound transmissions; Wherein, said sensed ultrasound transmissions are indicative of substance actually moved into said tissue, for determining the starting dose, the dose liberated from a transdermal delivery device or patch, and the remainder dose still within the transdermal delivery device or patch.

11. A transdermal delivery device or patch, with a built in sensor for determining the dose of a particular substance or drug remaining within the patch.

12. A transdermal delivery device or patch, with a connectable sensor for determining the dose of a particular substance or drug remaining within the patch.

13. A transdermal delivery device or patch for the delivery of insulin, constructed in the form of a Cap which is connected both to an ultrasonic delivery apparatus and a sensor for determining the dose of a particular substance or drug remaining within an absorbent pad contained within the patch-cap.

14. An array of transducers with both drug delivery and sensor capability, composed of 1 or more piezoelectric transducers designed to emanate an ultrasonic transmission through a transdermal delivery device or patch, and for receiving and interpreting the echo signal returning from contact with the drug laden patch, for determining the dose originally upon the patch, the dose liberated from the patch and the remaining dose on the patch.
